(19) United States
(12) Patent Application Publication
(10) Pub. No.: US 2021/0059585 A1
(43) Pub. Date: Mar. 4, 2021

(54) ON-CHIP INTEGRATED MULTI-WAVELENGTHS BIOLOGICAL SENSING DEVICE

(71) Applicant: NANOLAMBDA KOREA, Daejeong (KR)

(72) Inventors: Byung IL CHOI, Daejeon (KR); Cheng-Chun CHANG, Taipei (TW); Chien-Ta WU, Taipei (TW)

(21) Appl. No.: 17/001,902

(22) Filed: Aug. 25, 2020

Related U.S. Application Data
(60) Provisional application No. 62/891,601, filed on Aug. 26, 2019.

(51) Int. Cl. A61B 5/1455; H01L 31/02; H01L 31/0203; H01L 31/0216; H01L 31/173; A61B 5/00; G01N 21/31

(52) U.S. Cl. CPC A61B 5/14552; H01L 31/02019; H01L 31/0203; H01L 31/02162; G02B 5/20; A61B 5/7278; G01N 21/3103; G01N 21/314; H01L 31/173

(57) ABSTRACT

A chip-scale integrated multi-wavelength biological sensing device employing a plurality of filter-sensor assemblies is provided. The plurality of filter-sensor assemblies can include sufficient number of optical channels enabled by plasmonic filters having different nanoscale patterns and provided directly on an array of photodetectors. Combined with simultaneous illumination of light including multiple peak wavelengths with small full width at half maximum values, the independent optical channels of the plurality of filter-sensor assemblies enable correlation of detected optical signals with biological parameters. Signal processing methods for robustly extracting The PPG signals can be extracted by a robust signal processing method. Successful measurement of peripheral blood oxygen (SpO₂) and blood pressure has been demonstrated.

### RELATED APPLICATIONS

This application claims benefit of priority of U.S. Provisional Application No. 62/891,601 filed on Aug. 26, 2019, the entire contents of which is incorporated herein by reference.

### FIELD

The present disclosure is directed to a chip-scale integrated multi-wavelength photoplethysmography (PPG) sensing device and methods of operating the same.

### BACKGROUND

Multi-channel optical sensors refers to optical sensors that can measure not only the total intensity of incident light, but also an aspect of spectral distribution of the incident light. In this regard, a multi-channel optical sensor does not need to be able to provide a full analysis of the spectrum of incident light by generating the intensity distribution of light for a given wavelength range, i.e., by generating a curve representing the intensity of the light component within each wavelength interval. Such spectrometers are bulky and expensive, and are suitable for laboratories.

More practical approach to the multi-channel optical sensors is to detect light at multiple wavelength ranges. In this case, optical filters can be employed to limit the range of the wavelength range that is detected by a sensor. Typically, the optical filters do not provide 100% transmission or 100% blockage of light at any given wavelength, but generally provides a transmission response that measures a fraction of the incident light that is transmitted through the optical filter. The transmission response typically has a peak at a peak wavelength and a full width at half maximum that is on the order of 10%-20% of the peak wavelength as in the case of JENCOLOR® interference filters employed in AS73210 optical sensor provided by AMS AG™. Plasmonic filters tend to provide a full width at half maximum in a range from 8% to 10% of the peak wavelength as in the case of commercially available optical filters provided by Nanolambda™.

Multi-channel optical sensors integrated into a semiconductor chip provide distinctive advantages over bulky counterparts that are not based on a semiconductor chip in terms of size and cost. However, on-chip multi-channel optical sensors known in the art do not provide the level of spectral resolution that is necessary to provide an meaningful analysis of a biological sample. A rough metric for assessing the capability of a multi-channel optical sensor is the total number of different transmission responses of the optical filters, which correspond to the total number of independent optical parameters that can be generated from given incident light. This number is herein referred to as the number of channels. For example, if a multi-channel optical sensor includes three detectors with a red filter, a green filter, and a blue filter thereupon, respectively, a three-channel optical sensor is provided. The AS73210 sensor from AMS AG™ is an example of such a three-channel optical sensor. The measurement output generates a vector having a dimension of 3, the first element of the vector representing the magnitude of the output from the first sensor, the second element of the vector representing the magnitude of the output of the second sensor, and the third element of the vector representing the magnitude of the output of the third sensor. Multi-channel optical sensors with more than three channels is known in the art.

Unfortunately, the unit vectors from optical measurements do not correspond to a unit vector of a biological parameter. In other words, a biological sample rarely emits a single wavelength signal during a biological process, but emits a continuous spectrum of light instead. The convolution of an biological optical signal over a wide spectral range makes detection of a biological signal a very difficult task. Thus, detection of any biological signal requires much more than detection of a simple red, green, and blue spectrum that is typically provided by commercially available optical sensors. In other words, conventional multi-channel optical sensors that are suitable for color sensing does not generally generate enough optical data to enable meaningful biological measurements.

For the above reasons, conventional optical biological sensing devices employed a full-scale spectrometer capable of providing a full spectral analysis of light emitted from a biological sample. The spectrometer of such conventional optical biological sensing devices is a key component that enables the necessary spectral resolution, and typically a high cost mechanical component. Typically, optical gratings are employed to provide the necessary spectral resolution of light, and an array of optical sensors are employed. Generally, optical filters are not necessary in spectrometer-based biological sensing devices because the spectrometer decomposes the light and directs spectra of different wavelengths in different directions. However, a grating-based spectrometer is expensive and relatively bulky. For example, even a compact grating-based spectrometer such as STS-VIS available from Ocean Optics™ has a dimension of 40 mm×42 mm×24 mm. Further, grating-based spectrometers are mechanical components that are not compatible with semiconductor manufacturing process, and thus, cannot be integrated into a semiconductor die. Thus, conventional optical biological sensing devices employing a spectrometer tends to have a large volume and high cost, and cannot be integrated into a semiconductor die.

Notwithstanding the limitations of the conventional optical biological sensing devices, many useful applications have been found for conventional optical biological sensing devices. Photoplethysmography (PPG) is a subset of optical biological sensing methods. PPG is a method that collects light reflected or transmitted through skin so as to noninvasively monitor the pulsation of blood flow or blood composition in subcutaneous blood vessels. Since blood flow pulsations can reflect the operating conditions of the circulatory and respiratory systems of the human body, PPG signals can be used as indicators for many diseases, such as endothelial dysfunction, sympathetic neuropathy, cardiac arrhythmia, vasospasm, microcirculation, autonomic neuropathy, orthostatic hypotension, migraine, and peripheral artery disease. Due to the simple measurement structure, PPG sensing technology has been widely used in wearable devices to achieve heart rate detection in recent years. In 2018, the penetration rate of PPG sensing technology in wearable devices reached 98% and it is expected to reach 100% by 2020. It is expected that the global net profit of wearable devices will reach 52.5 billion U.S. dollars in 2024.

So far, PPG sensing devices using single-wavelength (SW) light sources have been the main stream on the market. Many studies have focused on reducing the effects of motion artifacts, mostly by using accelerometers to build up compensation signals, hence improving the signal-to-noise ratio (S/N) of the PPG signals. However, heart rate measurement error can be still up to 10% using the single-wavelength PPG (SW-PPG) sensing technology. Besides, the SW-PPG sensing technology may also suffer from many other factors during measurement, such as skin color, skin surface temperature and sensor contact pressure, resulting in a poor quality of PPG signals.

Accordingly, multi-wavelength PPG (MW-PPG) sensing technology has gradually attracted the attention of many scholars in recent years, and has been considered a robust PPG signal measurement method. In earlier studies, it has been noted that PPG sensing light sources at different wavelengths are recommended for the subjects with different skin colors. In applying multi-wavelength photoplethysmography (MW-PPG) sensing technology, the most suitable wavelength can be chosen to pick the PPG signals of best quality. This can effectively improve the accuracy of heart rate sensing by 15%. In the literature, experimental results show that under low-temperature conditions the blood perfusion decreases, resulting in the decrease of S/N of the PPG signal. While, if the MW-PPG sensing technology is applied, the best wavelengths can be selected for users at different skin temperatures, and hence the S/N of the PPG signal can be significantly increased by 50%. Besides, it is noted that wearing the PPG sensing devices will unavoidably cause contact pressure on the skin surface, which will cause different degrees of occlusion phenomenon for the microvascular and arterioles.

In other earlier studies, the inventors of the present disclosure pointed out that PPG sensing light sources at different wavelengths can be used at different degrees of occlusion phenomenon to achieve the best S/N of the PPG signals. By using integrated MW-PPG sensing devices, a better signal-to-noise ratio (S/N) of the PPG signals can be obtained. Besides, since PPG signals at different wavelengths can reflect the PPG signals measured from different depths of the body, the pulse transit time (PTT) derived from PPG signals at different wavelengths can also be obtained by using MW-PPG sensing technology. It was shown that the correlation coefficient R between the PTT and blood pressure can reach more than 0.9, demonstrating the feasibility of using MW-PPG signals for blood pressure measurement.

The general operational mode of the conventional MW-PPG is illustrated in FIG. 1A, spectrometers were used as the sensing devices for sensing MW-PPG signals. Not only does the large size of the conventional spectrometer lead to inconvenience in the measurement setup, but the high price of the conventional spectrometer also leads this approach unable to be on-chip integrated in the daily applications.

Another prior art approach to PPG is to use photodiodes (PDs) such as an AFE4404™ chip from Texas Instruments™, a BH1790GLC™ chip from ROHM Semiconductor™, or a MAX30102 chip from Maxim on-chip integrated™ to collect PPG signals of different wavelengths, as shown in FIG. 1B. Different light sources are sequentially activated, and the photodiode sequentially detects the light from a biological sample for each type of illumination. A same photodetector without an optical filter is employed to detect light reflected from a biological sample under different illumination conditions, which are generated by light emitting diodes emitting light at different wavelength sequentially. In other words, only one type of light emitting diodes emitting light at a respective peak wavelength is turned on at a time during this sequential measurement.

TABLE 1
Comparison of prior art MW-PPC devices

| Prior | | | | | |
|---|---|---|---|---|---|
| art | | Number | Measure- | | |
| device | Sensor Chip | of | ment | | |
| number | (Manufacturer) | Channels | Mode | Cost | Size |
| 1 | AFE4404 | 3 | Sequential | Low | Small |
| | (Texas Instruments) | | | | |
| 2 | BH1790GLC | 1 | Sequential | Low | Small |
| | (ROHM Semiconductor) | | | | |
| 3 | MAX30102 | 2 | Sequential | Low | Small |
| | (Maxim on-chip integrated) | | | | |

The sequential illumination approach employing a photodetector can effectively reduce the size and cost of the integrated MW-PPG sensing devices. However, for example, to acquire N different wavelength PPG signals through this sequential sampling architecture in which the PPG signals are acquired one at a time, not only will the sampling rate of PPG signals at each wavelength be reduced by 1/N, but it will also require light sources of N different wavelengths. When N is large, this architecture would become difficult in implementation. For practical considerations, only N=2 or 3 are implemented in general.

### SUMMARY

Multi-wavelength photoplethysmography (MW-PPG) employing a full-scale spectrometer has been known to provide superior measurement to signal-wavelength photoplethysmography (SW-PPG). However, the spectrometer of a MW-PPG is expensive and bulks, and thus, was not affordable or portable. The present disclosure provides a chip-scale integrated multi-wavelength biological sensing device employing a plurality of filter-sensor assemblies. The plurality of filter-sensor assemblies provide the function of a combination of a spectrometer and an array of optical sensors in full-scale MW-PPG apparatus at a significantly low cost while providing portability. The plurality of filter-sensor assemblies can include sufficient number of optical channels enabled by plasmonic filters having different nanoscale patterns and provided directly on an array of photodetectors. Combined with simultaneous illumination of light including multiple peak wavelengths with small full width at half maximum values, the independent optical channels of the plurality of filter-sensor assemblies enable correlation of detected optical signals with biological parameters. Signal processing methods for robustly extracting The PPG signals can be extracted by a robust signal processing method. Successful measurement of peripheral blood oxygen (SpO₂) and blood pressure has been demonstrated.

According to an aspect of the present disclosure, an on-chip integrated multi-wavelength biological sensing device comprising an electronic package, wherein the electronic package comprises: a light source assembly configured to emit light having multiple peak wavelengths simultaneously toward a biological sample; a plurality of filter-sensor assemblies configured to measure spectral distribution of incident light that impinges from the biological sample, wherein each of the filter-sensor assemblies comprises a respective optical filter providing a respective optical transmission response and a respective optical sensor; an embedded processor and a memory unit embedded within at least one semiconductor chip, wherein the memory unit stores an automated program configured to compile the measured spectral distribution of the incident light and to generate a measurement value for at least one biological measurement parameter pertaining to the biological sample; and an enclosure containing the light source assembly, the plurality of filter-sensor assemblies, and the semiconductor chip. A biological parameter such as blood pressure and/or an oxygen saturation level in oxygen-carrying cells of a person may be measured as the at least one biological measurement parameter.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1A illustrates a prior art multi-wavelength photoplethysmography (MW-PPG) sensing device that employs a broad-band light source and a conventional spectrometer that separates incident light by diffraction.

FIG. 1B illustrates a prior art MW-PPG device that employs sequential sampling of signals in which a target is sequentially irradiated with light beams from multiple light sources one beam at a time during measurement.

FIG. 1C illustrates an on-chip integrated MW-PPG device of the present disclosure in which multiple light beams are simultaneously irradiate a target and multiple photoplethysmography signals are generated simultaneously employing a chip-scale PPG sensor on-chip integrated into a sensing device according to an embodiment of the present disclosure.

FIG. 2 schematically illustrates the schematics of a mathematical model employed for the on-chip integrated MW-PPG device of the present disclosure.

FIG. 3 is a flowchart illustrating the signal processing steps for extracting robust maximal-ratio combined (MRC)-MW-PPG signals.

FIG. 4 illustrates signal processing steps for SpO₂ measurement using the integrated MW-PPG sensing device of the present disclosure.

FIG. 5 illustrates signal processing steps for blood pressure measurement using the integrated MW-PPG sensing device of the present disclosure

FIG. 6A is a photo of an example of on-chip integrated MW-PPG device of the present disclosure.

FIG. 6B is measured spectra of the multi-wavelength light emitted from the light source of the on-chip integrated MW-PPG device of the present disclosure.

FIG. 7A illustrates steps for operating the on-chip integrated multi-wavelength biological sensing device of the present disclosure.

FIG. 7B is a schematic of the on-chip integrated multi-wavelength biological sensing device of the present disclosure.

FIGS. 8A-8D illustrate waveform and variation of the PPG signals over multiple measurements. FIGS. 8A and 8C show the relative signal intensity in multiple measured data as a function of wavelength measured by a reference single wavelength PPG device. FIGS. 8B and 8D show the relative signal intensity in multiple measured data as measured by the on-chip integrated MW-PPG device of the present disclosure.

FIG. 9 shows comparison of signal variations between a prior art single-wavelength PPG (SW-PPG) and the MW-PPG of the present disclosure over 10 subjects. The data for the SW-PPG was generated employing a prior art signal-wavelength photoplethysmography (SW-PPG) sensing device. The data for the MW-PPG of the present disclosure was generated employing a manufactured sample of the MW-PPG device of the present disclosure.

FIG. 10 illustrates a correlation analysis between SpO₂ and R-values extracted from the on-chip integrated MW-PPG device of the present disclosure.

FIG. 11A illustrates a correlation analysis between the blood pressure measured by the reference instrument against the extracted from the developed integrated MW-PPG sensing device for systolic blood pressure (SBP).

FIG. 11B illustrates a correlation analysis between the blood pressure measured by the reference instrument against the extracted from the developed integrated MW-PPG sensing device for diastolic blood pressure (DBP).

FIG. 12A illustrates a filter response for five filters for blue light that were employed in the fabricated sample of the on-chip integrated MW-PPG device of the present disclosure.

FIG. 12B illustrates a filter response for five filters for green light that were employed in the fabricated sample of the on-chip integrated MW-PPG device of the present disclosure.

FIG. 12C illustrates a filter response for five filters for infrared light that were employed in the fabricated sample of the on-chip integrated MW-PPG device of the present disclosure.

### DETAILED DESCRIPTION

The various embodiments will be described with reference to the accompanying drawings. Elements are not drawn to scale. Wherever possible, the same reference numbers will be used throughout the drawings to refer to the same or like parts. References made to particular examples and implementations are for illustrative purposes, and are not intended to limit the scope of the invention or the claims.

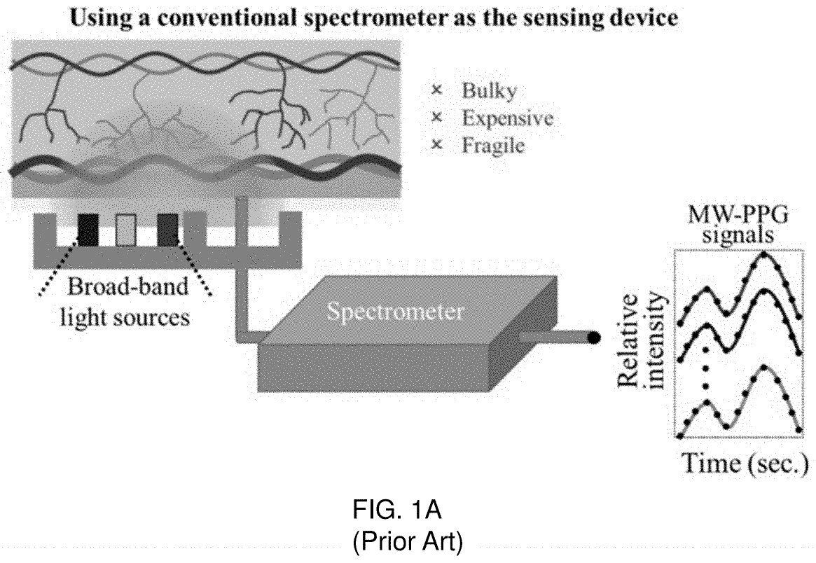

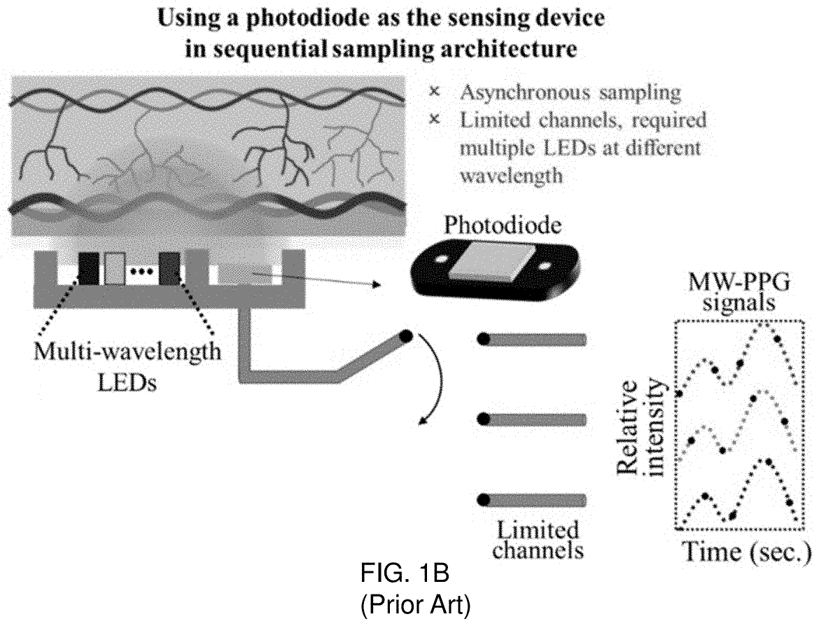

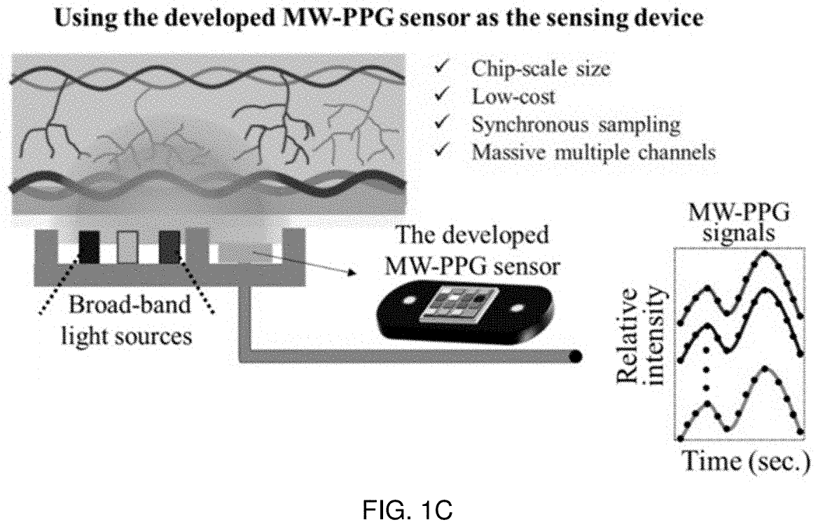

FIG. 1C illustrates an on-chip integrated multi-wavelength biological sensing device of the present disclosure. As used herein, an “on-chip” device refers to an electronic device including all main components in the form of at least one semiconductor chip, i.e., a semiconductor die. An on-chip device differs from non on-chip devices in that the on-chip devices does not include any key component that is not attached to an assembly including a semiconductor die or a plurality of semiconductor dies connected to a circuit board. A peripheral device such as a universal serial bus (USB) may be optionally attached to an on-chip device. However, such a peripheral device is not a key component of such an on-chip device. As such, on-chip devices are portable and may be attached to a computing device such as a personal computer or a cellular phone. As used herein, an “integrated sensing device” refers to a sensing device including an entire set of components needed to perform a measurement within the sensing device. If a measurement requires a light source, such a light source is integrated into the sensing device. As used herein, a “multi-wavelength” device refers to a device that employs a light source emitting a light spectrum containing multiple peak wavelengths. In other words, the spectrum of emitted light includes multiple peak wavelengths. A “biological sensing device” refers to a sensing device that can measure a biological parameter, i.e., a parameter related to a biological aspect of a biological sample such as a human being. As such, an “integrated multi-wavelength biological sensing device” refers to a integrated sensing device configured for measurement of a biological parameter, embodied as electronic device including all main components in the form of at least one semiconductor chip, and including a light source emitting a light spectrum containing multiple peak wavelengths.

The integrated multi-wavelength biological sensing device of the present disclosure illustrated in FIG. 1C may be an on-chip integrated multi-wavelength photoplethysmography (MW-PPG) sensing device. However, the integrated multi-wavelength biological sensing device of the present disclosure illustrated in FIG. 1C differs from a non-on-chip MW-PPG apparatus of FIG. 1A in that the spectrometer of FIG. 1A is not an electronic component that can be manufactured as a semiconductor chip. As a non-electronic component, the spectrometer in the MW-PPG apparatus of FIG. 1A cannot be formed as a semiconductor die, and cannot provide an on-chip device. Typically, semiconductor chips are formed on a semiconductor substrate such as a silicon substrate or a III-V compound semiconductor substrate, and has maximum lateral dimensions not greater than 2.5 cm×2.5 cm.

The integrated multi-wavelength biological sensing device of the present disclosure illustrated in FIG. 1C differs from an on-chip single-wavelength photoplethysmography (SW-PPG) device of FIG. 1B in that the light source emits light with a single peak wavelength at any given time. Light emitting diodes having different peak wavelengths can be turned on sequentially such that only a single peak wavelength is present within the incident beam that is directed to a biological sample. In other word, the light spectrum of the incident beam always has a single peak wavelength, although the peak wavelengths may change over time as different sets of light emitting diodes are turned on sequentially. Such SW-PPG measurement may be sufficient to monitor a time-dependent parameter such as a heart rate of a person. In contrast, the light emitting diodes of the integrated multi-wavelength biological sensing device of the present disclosure are turned on simultaneously, and therefore, the light spectrum from the light source in the integrated multi-wavelength biological sensing device of the present disclosure has multiple peaks, such as three peaks or more.

Illumination of light having multiple peaks in the light spectrum can be disadvantageous from the viewpoint of signal analysis because data is not generated under different illumination conditions. In other words, the SW-PPG scheme illustrated in FIG. 1B can sequentially illuminate red, green, and blue beam on a biological sample, and collect measurement data from multiple channels including a respective interference filter (such as 1-3 channels as discussed above). Each measurement cycle including a full cycle of light emitting diode illumination generates a vector representing the measured intensity of light such that the vector has a dimension corresponding to the product of the number of measurement channels (each having a respective optical filter) and the total number of different peak wavelengths within the sequentially activated array of light emitting diodes.

When light having multiple peaks in the light spectrum is illuminated, cycling through different light emitting diodes is not employed. In other words, all light emitting diodes having different peak wavelengths are turned on simultaneously. Thus, the dimension of the vector representing the number of datapoints for a single measurement is the same as the number of measurement channels, which is the number of optical filters that generate different light filtering characteristics, i.e., different transmission responses.

According to an aspect of the present disclosure, a sufficient number of optical filters capable of discriminating the optical spectrum of light from a biological sample is employed to increase the number of meaningful optical data. The set of optical filters are selected such that full width at half maximum is small relative to the value of the peak wavelength. In one embodiment, plasmonic filters employing a metallic film including nanoscale patterns is employed to provide optical filters that can provide a large number of sufficiently optically distinguishable transmission responses. The increased number of channels enable correlation of measured optical spectrum with biological properties. In a demonstrated example, the integrated multi-wavelength biological sensing device of the present disclosure illustrated in FIG. 1C was employed as an MW-PPG device including 15 channels, i.e., including 15 different optical filters.

Illumination of light having multiple peaks in the light spectrum from the integrated multi-wavelength biological sensing device of the present disclosure eliminates the need to cycle through different sets of light emitting diodes. Thus, all channels of the integrated multi-wavelength biological sensing device of the present disclosure can continuously generate data corresponding to a same measurement condition without a gap in time, and thus, increases the temporal coverage and sensitivity of the measurement.

The inventors of the present disclosure manufactured and tested an integrated MW-PPG sensing device as an example of the integrated multi-wavelength biological sensing device of the present disclosure. The integrated MW-PPG sensing device developed measured only several square micrometers before packaging. The size of the package had to be increased beyond the size of the sensor itself to accommodate a biological sample. The integrated MW-PPG sensing device was compact, robust, and lightweight. To provide a large number of PPG signals at different wavelengths, only one broad spectrum light-emitting diode (LED) or a few LEDs covering broad spectrum are required. Furthermore, signal processing algorithms were developed to robustly extract PPG signals using this developed integrated MW-PPG sensing device. Experimental results show that the S/N of the maximal-ratio combined (MRC) MW-PPG signals, namely MRC-MW-PPG signals, can be increased by up to 50%, compared to those acquired from the conventional single wavelength approach. Besides, the inventors were able to successfully demonstrate simultaneous heart rate measurement, SpO₂ measurement and blood pressure measurement using this integrated MW-PPG sensing device developed.

While the present disclosure is described employing an example in which an on-chip integrated multi-wavelength biological sensing device comprises an on-chip integrated MW-PPG sensing device, it is understood that the on-chip integrated multi-wavelength biological sensing device of the present disclosure may be applied to any other biological measurements. Such general applications are expressly contemplated herein.

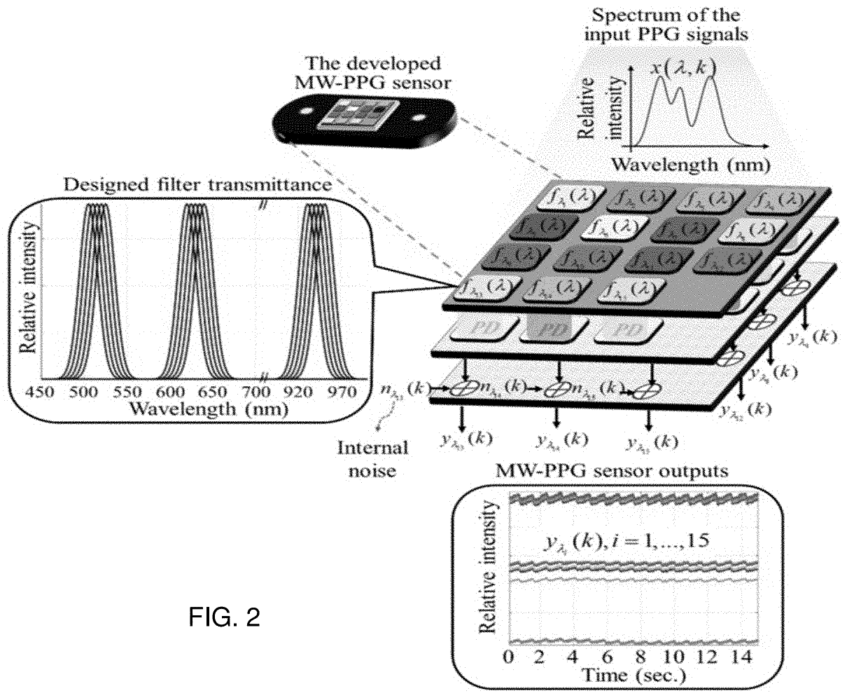

Referring to FIG. 2, general hardware design of an exemplary on-chip integrated MW-PPG sensing device is schematically illustrated. The core technology of this sensor is based on plasmonic filters which can be on-chip integrated onto a regular photo-detector such as a complementary metal-oxide-semiconductor (CMOS) imager. By introducing nanoscale structures on metal films, plasmonic filters can provide a unique way to control polarization and wavelength of light passing through the structures. One of the significant differences of the plasmonic filters is that the transmission wavelength can be controlled only by the lateral structures on a single layer. This makes it possible to produce a device containing different filter channels in a cost-effective manner. The single layer plasmonic metal structures can be monolithically fabricated using the standard semiconductor wafer process such as nanoimprint lithography and etching processes, which enables the low manufacturing cost for the volume applications. The fabrication cost of the plasmonic filters can be as low as a few dollars at volume, which is one of the most advanced processes in making on-chip spectrometers.

The inventors utilized the same concept, but made a chip-scale integrated MW-PPG sensing device to synchronously detect MW-PPG signals at 15 wavelengths, including: 505 nm, 510 nm, 515 nm, 520 nm, 525 nm, 620 nm, 625 nm, 630 nm, 635 nm, 640 nm, 930 nm, 935 nm, 940 nm, 945 nm, and 950 nm. The 15 wavelengths were grouped into three regions with three major peak wavelengths: 515 nm, 630 nm and 940 nm. By using the red region PPG signals centered at 630 nm and the infrared region PPG signals centered at 940 nm, the R-values could be obtained for the SpO₂measurement. Furthermore, by using the green region PPG signals centered at 515 nm and the infrared region PPG signals centered at 940 nm, the PTT could be extracted for the blood pressure measurements.

The subsequent section describes the mathematical model of the integrated MW-PPG sensing device of the present disclosure, and the signal processing algorithms for obtaining robust PPG signals, SpO₂ and blood pressure. This section describes how the raw MW-PPG signals, the PTTs of the raw MW-PPG signals, and the PTT-compensated PPG signals are extracted from the integrated MW-PPG sensing device the inventors developed. These quantities will be used in the sequel for robust PPG measurement, SpO₂ measurement, and blood pressure measurement. Let x(k, k) denote the spectrum reflected from tissues emitted by the the designed light source assembly, as shown in FIG. 2.

Assume x(λ, k) is shining into the developed chip-scale integrated MW-PPG sensing device, where k is the discrete time index. Let fᵢ(λ) be the transfer function of the i-th filter in the developed chip-scale integrated MW-PPG sensing device. The raw PPG signals from the i-th filter can be represented as:

yᵢ(k)=sᵢ(k)+nᵢ(k), i=1, . . . , 15: Equation (1)

, where sᵢ(k)=∫fᵢ(λ)x(λ,k)dλ signal component, nᵢ(k) is Gaussian noise component, and y₁(k), . . . , y₁₅(k) are the raw PPG signals at wavelengths 505 nm, 510 nm, 515 nm, 520 nm, 525 nm, 620 nm, 625 nm, 630 nm, 635 nm, 640 nm, 930 nm, 935 nm, 940 nm, 945 nm, and 950 nm, respectively. In the design, 505 nm PPG signal y₁(k) is used as a reference, and the PTT of the i-th PPG signal is expressed as:

$${PTT}_{i} = ( \frac{1}{{f}_{s}} ) {argmax}_{τ ∈ Z} ( Corr ( {y}_{1} ( k ) , {y}_{i} ( k ) , τ ) ) , i = 1 , … , 15. \qquad Equation ( 2 )$$

where Corr(y₁(k), yᵢ(k), τ)=Σy*₁(k)yᵢ(k+τ), i=1, . . . , 15 is the cross-correlation function between y1(k) and yi(k), τ is the discrete index displacement, and fₛ is the sampling rate of the developed integrated MW-PPG sensing device.

As known in the art, skin is a layer structure and blood vessels are located in different layers, for example, small arteries are located in hypodermis layer which is the innermost layer of skin, arterioles are located in dermis layer and capillaries are located in the epidermis layer. When the blood pulse generated by the heart, it will arrive at small arteries, arterioles, and capillaries in order at different times. Since light with different wavelengths can penetrate into different depths of skin, MW-PPG signals at different wavelengths reflect the signals probing to different depths of blood vessels. In other words, MW-PPG signals carry the information of pulse arrival time at different depths of blood vessels. Conventionally, pulse transit time (PTT) is considered to be the time delay between the peak of PPG signals against the R peaks of electrocardiogram (ECG) signals. In this work, the pulse transit time (PTT) is defined as the time shifting between MW-PPG signals at different wavelengths, also known as local PTT. The PTT-compensated PPG signals are then expressed as:

[symbol]

(k)= [symbol] (k)+ [symbol] (k), i=1, . . . , 15: Equation (3)

, where [symbol] (k)=sᵢ(k−fₛPTTᵢ) and [symbol] (k)=nᵢ(k−fₛPTTᵢ).

Extraction robust PPG signals, SpO₂and blood pressure measurement can be performed in the following manner. First, the MRC algorithm for deriving robust PPG signals from the MW-PPG signals is presented. Then, the method of obtaining R-values from the PTT-compensated MW-PPG signals for SpO₂ measurement is introduced. Third, the method of using PTTi for blood pressure measurement is explained.

Combination of MW-PPG signals for extracting of robust PPG signals can be performed in the following manner. Assume the MW-PPG signals from the developed integrated MW-PPG sensing device is quasi-steady, where E[s̅ᵢ²(k)]=\tilde{s}ᵢ²(k). Assume the noise of the i-th filter is Gaussian with zero-mean and variation σᵢ². The signal-to-noise ratio S/N of the i-th filter can be defined as:

$$S \text{/} {N}_{i} = \frac{E [ {\tilde{s}}_{i}^{2} ( k ) ]}{E [ {\tilde{n}}_{i}^{2} ( k ) ]} = \frac{{\tilde{s}}_{i}^{2} ( k )}{{σ}_{i}^{2}} \qquad Equation ( 4 )$$

Assume the weights to the MW-PPG signals at different wavelengths are ωi, i=1, . . . , 15. The MRC-MW-PPG signal from the PTT-compensated MW-PPG signals can be expressed as

$$\overline{y} ( k ) = {\sum }_{i = 1}^{15} {w}_{i} {\tilde{y}}_{{λ}_{i}} ( k ) = \overline{s} ( k ) + \overline{n} ( k ) , where \overline{s} ( k ) = {\sum }_{i = 1}^{15} {w}_{i} \tilde{{s}_{i}} ( k ) , and \overline{n} ( k ) = {\sum }_{i = 1}^{15} {w}_{i} \tilde{{n}_{i}} ( k ) . \qquad Equation ( 5 )$$

It can be assumed that the signal power and noise power of the MRC-MW-PPG signals can be expressed respectively as:

$$\{ \begin{matrix}E [ {\overline{s}}^{2} ( k ) ] = {E ( {\sum }_{i = 1}^{15} {w}_{i} \tilde{{s}_{i}} ( k ) )}^{2} = {( {\sum }_{i = 1}^{15} {w}_{i} \tilde{{s}_{i}} ( k ) )}^{2} \\ E [ {\overline{n}}^{2} ( k ) ] = {E ( {\sum }_{i = 1}^{15} {w}_{i} \tilde{{n}_{i}} ( k ) )}^{2} = {\sum }_{i = 1}^{15} {w}_{i}^{2} {σ}_{i}^{2}\end{matrix} . \qquad Equation ( 6 )$$

Therefore, the S/N of the MRC-MW-PPG signals S/Nₜₒₜₐₗ is defined as

$${( {\sum }_{i = 1}^{15} {w}_{i} \tilde{{s}_{i}} ( k ) )}^{2} \text{/} ( {\sum }_{i = 1}^{15} {w}_{i}^{2} {σ}_{i}^{2} ) .$$

According to the well-known Cauchy-Schwarz inequality and the MRC signal combination algorithm, it can be shown that:

$$S \text{/} {N}_{total} \leq {\sum }_{i = 1}^{15} S \text{/} {N}_{i} . \qquad Equation ( 7 )$$

S/Nₜₒₜₐₗ can be maximized at

$$S \text{/} {N}_{total} = {\sum }_{i = 1}^{15} S \text{/} {N}_{i}$$

with the optimal weights

$${w}_{i}^{*} = \frac{\tilde{{s}_{i}} ( k )}{{σ}_{i}^{2}}$$

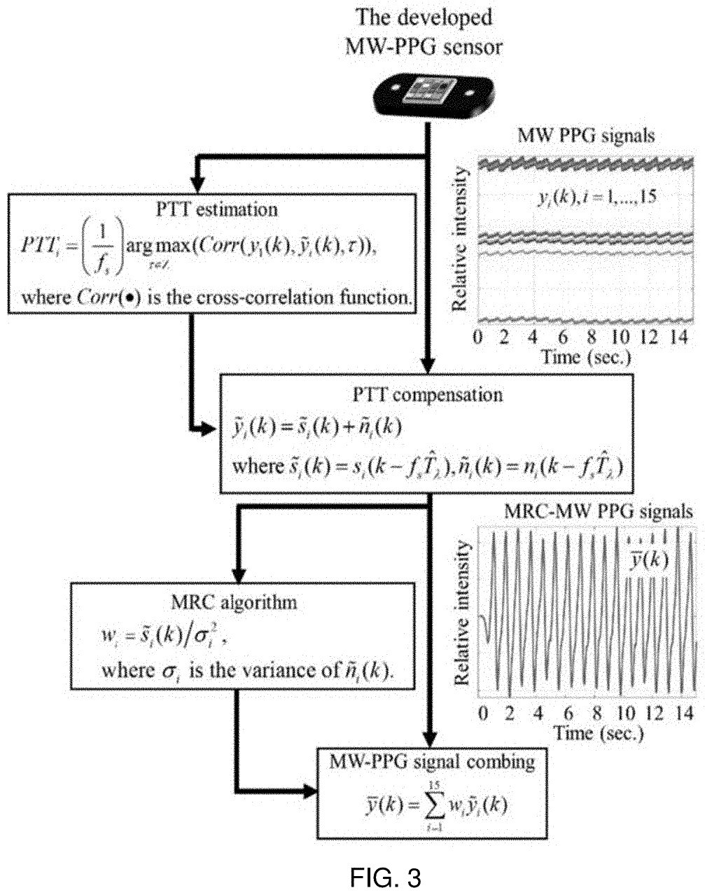

The flowchart shown in FIG. 3 summarizes the algorithms for obtaining the robust MRC-MW-PPG signals. The computational cost of the MRC algorithm is low and is linear scaling with respective to the number of selected components. In other words, the computational complexity of the MRC algorithm implemented was O(n), where n is the number of the picked wavelengths on the multi-wavelength PPG signals acquired by the developed integrated MW-PPG sensing device.

The following section describes MW-PPG signal processing methods for SpO₂ measurement. SpO₂ is defined as the measurement of the amount of oxygen dissolved in blood. Light at different wavelengths can be used to probe the absorption level of Oxygen-bound Hemoglobin (HbO₂) and Hemoglobin (Hb). It has been widely reported that the attenuations by Hb and HbO₂ are largely different at wavelength 660 nm, and are nearly the same at 940 nm. In other words, if using the signal at 940 nm as a normalizer, the absorption level can be clearly distinguished by watching the signal at 660 nm. 660 nm and 940 nm are then widely used for SpO₂ measurement in the research fields as well as in industries. From Equation (3), it is noted that \tilde{y}₈(k) and \tilde{y}₁₃(k) are the PPG signals at 660 nm and 940 nm, respectively. According to the Beer-Lambert law, the optical density (OD) of \tilde{y}₈(k) and \tilde{y}₁₃(k) can be defined respectively as:

$$\{ \begin{matrix}{OD}_{8} = {\int }_{0.25}^{2.5} {\tilde{ϒ}}_{8} ( f ) df \text{/} {\tilde{ϒ}}_{8} ( 0 ) \\ {OD}_{13} = {\int }_{0.25}^{2.5} {\tilde{ϒ}}_{13} ( f ) df \text{/} {\tilde{ϒ}}_{13} ( 0 )\end{matrix} \qquad Equation ( 8 )$$

where \tilde{Y}ᵢ(f)= [symbol] [ [symbol] (k)] is the frequency response of the i-th PPG signal, and [symbol] [●] is a Fourier transform. The R-values can be associated by R=OD₈/OD₁₃. SpO₂ can be approximated by:

SpO₂=aR+b: Equation (9)

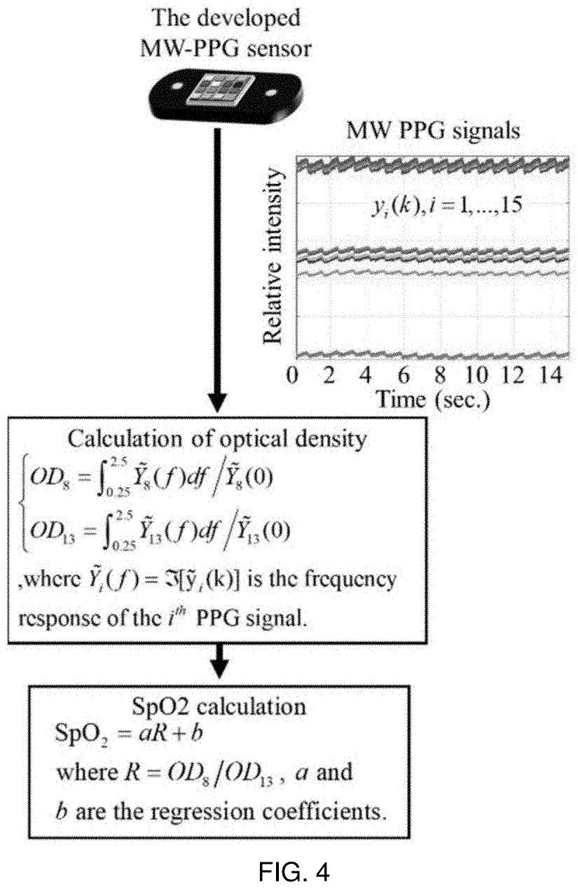

, where, a and b are regression coefficients of the linear models. The signal processing procedure of SpO₂ measurement using the developed integrated MW-PPG sensing device is summarized in FIG. 4.

The following section describes MW-PPG signals processing methods for blood pressure measurement. The PTTᵢ of Equation (2) can have a high correlation with diastolic blood pressure (DBP) and systolic blood pressure (SBP). The relationship of PTTᵢ and blood pressure can be established by using a linear regression model.

In this work, the averaged PTT can be computed by:

$${PTT}_{avg} = \frac{1}{15} {\sum }_{i = 1}^{15} {PTT}_{i} .$$

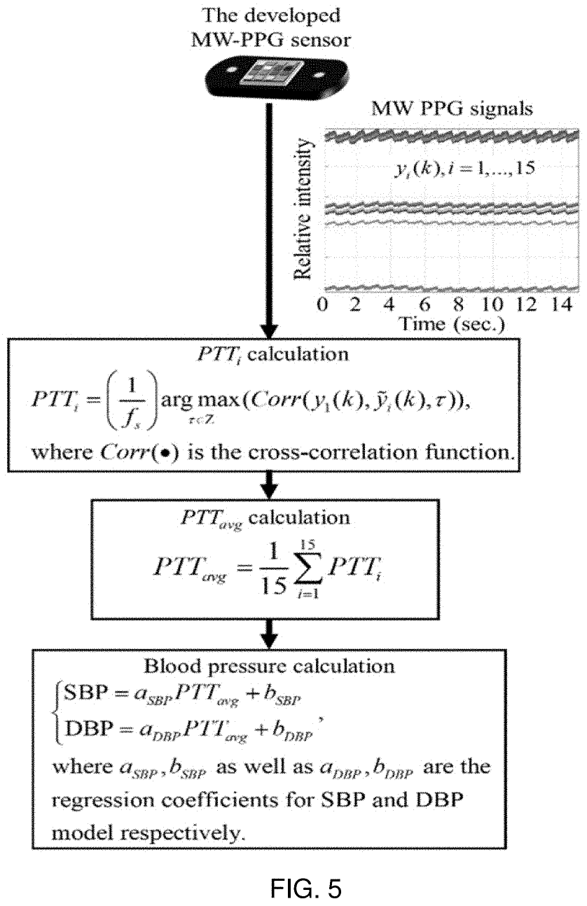

PTT_{avg} can be associated with DBP as well as PTT_{avg} with SBP as follows:

$$\{ \begin{matrix}SBP = {a}_{SBP} {PTT}_{avg} + {b}_{SBP} \\ DBP = {a}_{DBP} {PTT}_{avg} + {b}_{DBP}\end{matrix} , \qquad ( 10 )$$

where a_{SBP}, b_{SBp} and a_{DBP}, b_{DBP} are the regression coefficients of the linear models for DBP and SBP, respectively. The signal processing procedure of SBP and DBP measurement using the developed integrated MW-PPG sensing device is summarized in FIG. 5.

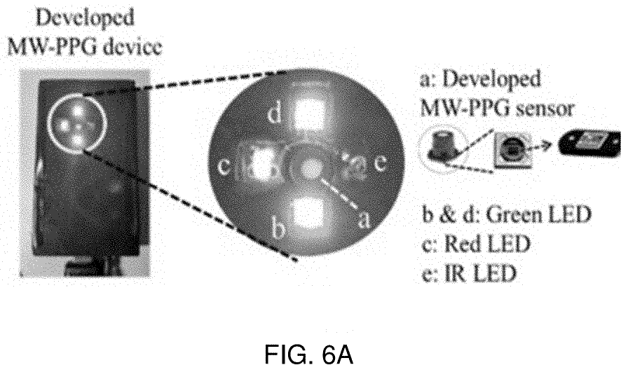

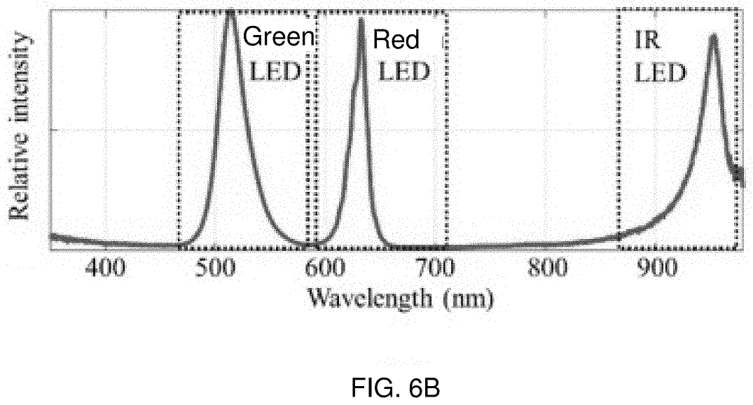

Referring to FIG. 6A, a photo of an exemplary on-chip integrated MW-PPG sensing device of the present disclosure is shown. Part a corresponds to a plurality of filter-sensor assemblies configured to measure spectral distribution of incident light that impinges from a biological sample. Parts b and d are green light emitting diodes with a peak wavelength at 515 nm. Parts c and e are red and infrared light emitting diodes with peak wavelengths at 630 nm and 940 nm, respectively.

FIG. 6B shows the light spectrum generated by simultaneously turning on all the light emitting diodes as measured by a spectrometer model USB4000™ by Ocean Optics™. The light spectrum shown in represents the light spectrum that impinges on a biological sample for measurement employing the on-chip integrated multi-wavelength biological sensing device.

The functionalities of the developed chip-scale integrated MW-PPG sensing device, have been generally verified. For the purposes of verification, the inventors only acquired 10 subjects, whose ages ranged from 20 to 60 and the ratio of men to women was 7:3, with males ranging from 160 to 180 centimeters in height and females ranging from 155 to 170 centimeters in height. To demonstrate the advantages of the chip-scale integrated MW-PPG sensing device developed, a SW-PPG sensor representing a conventional signal-wavelength PPG detector was used as a reference device. To compare the stability of the PPG signals, each subject was asked to use both the integrated MW-PPG sensing device developed and the SW-PPG sensor to acquire 15 second signals. Also, to conduct a correlation analysis between the SpO₂ and the R-values extracted from the developed integrated MW-PPG sensing device, a blood oximetry meter (TRUST, TD-8250A) was used as a reference instrument. Besides, to perform the correlation analysis between SBP, DBP against the PTT_{avg} extracted from the developed chip-scale integrated MW-PPG sensing device, an upper arm blood pressure monitor (Omron, HEM-7121) was used as the reference instrument. It is worth mentioning that while considering the frequency of the human heart rate pulse signal is normally around 0.25-2.5 (Hz), the inventors used Parks-McClellan algorithm to design a 64-degree band-pass filter (BPF), with a passband of 0.3-4.0 Hz, to eliminate the out of band noise.

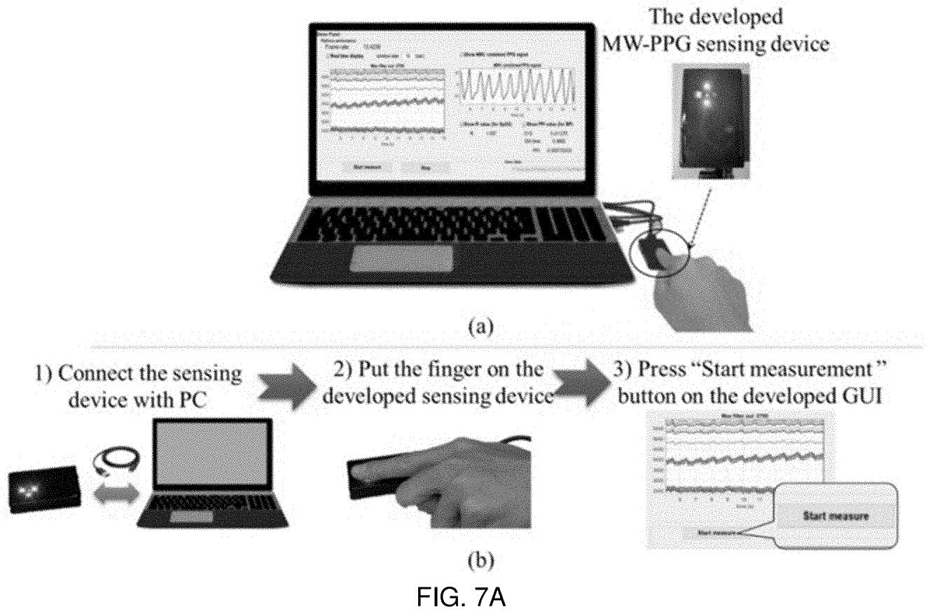

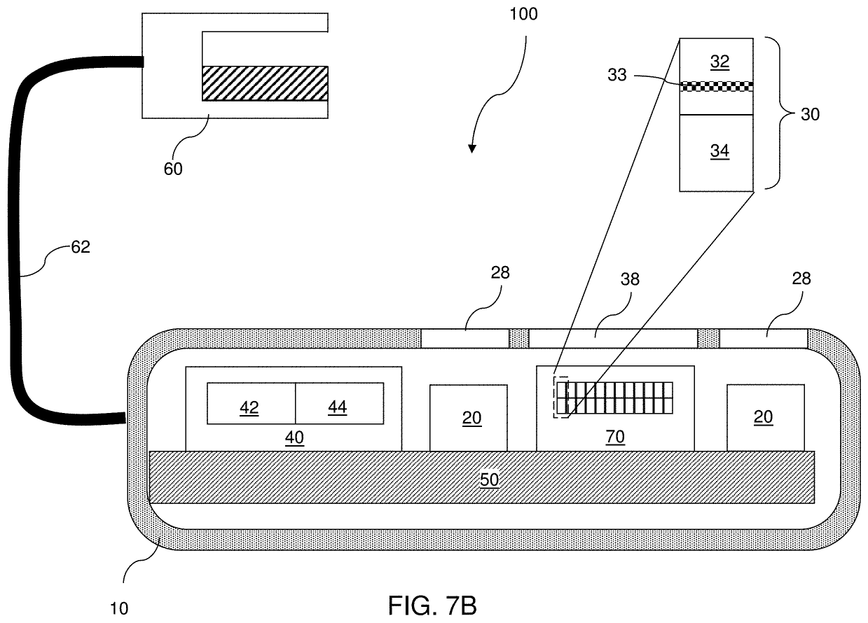

FIG. 7A shows an exemplary procedure for operating the on-chip integrated multi-wavelength biological sensing device of the present disclosure. To collect multi-wavelength biological signals (i.e., biological signals generated under the condition of illumination of light including multiple peak wavelengths), the on-chip integrated multi-wavelength biological sensing device can be connected to a host computing device such as a personal computer (PC) or a cellular phone employing a universal serial bus (USB) cable or via wireless connection. For measurement, an index finger of a person can be placed on the on-chip integrated multi-wavelength biological sensing device. Measurement can be initiated, for example, by pressing a “start measurement” button on the graphical user interface (GUI). In the exemplary on-chip integrated multi-wavelength biological sensing device constructed and tested by the inventors, the GUI interface was based on the MATLAB™ R2017a platform in this example. During testing of the 15 seconds of raw MW-PPG signals yi(k), i=1, . . . , 15yi(k), i=1, . . . , 15 was recorded. By using Equations (2) and (3), each PPG signals' PTT PTTᵢ, i=1, . . . , 15PPTi, i=1, . . . , 15 and each PTT-compensated PPG signal \tilde{y}ᵢ(k), i=1, . . . , 15 can be extracted from yi(k), i=1, . . . , 15yi(k), i=1, . . . , 15. Besides, the MRC-MW-PPG signal y(k)y (k) can be obtained from \tilde{y}ᵢ(k), i=1, . . . , 15 using the presented MRC signal combining the algorithm described above. Besides, the R-values are calculated from the 660 nm and 940 nm PPG signal, \tilde{y}₈(k) and \tilde{y}₁₃(k), based on the algorithm discussed above. Also, the PTTavg is calculated from PTTᵢ, i=1, . . . , 15 according to the algorithm discussed above.

Referring to FIG. 7B, a general schematic for the on-chip integrated multi-wavelength biological sensing device 100 of the present disclosure is illustrated. The on-chip integrated multi-wavelength biological sensing device 100 such as the on-chip integrated multi-wavelength photoplethysmography (MW-PPG) can be embodied in an electronic package, i.e., a package including electronic components and is capable of interfacing with computing devices such as personal computers or cellular phones, for example, via a universal serial bus (USB) cable 62 and a USB connector 60. According to various embodiments of the present disclosure, an on-chip integrated multi-wavelength biological sensing device 100, an an electronic package is provided. The electronic package comprises: a light source assembly 20 configured to emit light having multiple peak wavelengths simultaneously toward a biological sample (such as a body part of a human being); a plurality of filter-sensor assemblies 30 configured to measure spectral distribution of incident light that impinges from the biological sample, wherein each of the filter-sensor assemblies 30 comprises a respective optical filter 32 providing a respective optical transmission response and a respective optical sensor 34; an embedded processor 42 and a memory unit 44 embedded within at least one semiconductor chip 40, wherein the memory unit 44 stores an automated program configured to compile the measured spectral distribution of the incident light and to generate a measurement value for at least one biological measurement parameter pertaining to the biological sample; and an enclosure 10 containing the light source assembly 20, the plurality of filter-sensor assemblies 30, and the semiconductor chip 40.

Each of the optical sensors 34 may be a semiconductor sensor formed on a detector semiconductor chip 70. In one embodiment, the plurality of filter-sensor assemblies 30 may be embodied as electronic components within the detector semiconductor chip 70, which can include a CMOS circuitry for converting the optical input to the plurality of filter-sensor assemblies 30 to digital signals that are transmitted to the embedded processor 42 via the circuit board 50.

In one embodiment, the on-chip integrated multi-wavelength biological sensing device 100 comprises a circuit board 50 to which the light source assembly 20, the plurality of filter-sensor assemblies 30, the embedded processor, and the memory unit are attached.

In one embodiment, the plurality of filter-sensor assemblies 30 is configured to synchronously measure the spectral distributions for each of the filter-sensor assemblies 30. In one embodiment, the plurality of filter-sensor assemblies 30 can continuously measure the biological sample without interruption of measurement.

In one embodiment, the light source assembly 20 comprises a plurality of light emitting diodes configured to emit light of different peak wavelengths simultaneously. In one embodiment the total number of the plurality of filter-sensor assemblies 30 is greater than the total number of the different peak wavelengths of the plurality of light emitting diodes at least by a factor 3.In other words, at least three, such as 4-12, filter-sensor assemblies 30 may be provided per peak wavelength of light contained in the illuminating light spectrum that is emitted from the light source assembly 20 and continuously illuminates the biological sample.

In one embodiment, the electronic package comprises at least one emission window pane 28 and a reception window pane 38. The plurality of light emitting diodes is configured to emit light simultaneously through a respective one of the at least one emission window pane 28; and the plurality of filter-sensor assemblies 30 is configured to receive light through the reception window pane 38.

In one embodiment, the electronic package comprises at least as many emission window panes as the total number of peak wavelengths among the multiple peak wavelengths. Each light emitting diode among the plurality of light emitting diodes is configured to emit light through different emission window panes. The emission window panes are arranged around the reception window pane on a front side of the electronic package.

In one embodiment, the optical filters 32 within the plurality of filter-sensor assemblies 30 comprise plasmonic filters including a respective metallic film 33 containing nanoscale structures. Plasmonic filters are described in U.S. Pat. No. 10,578,486 titled “Method of calibrating spectrum sensors in a manufacturing environment and an apparatus for effecting the same,” U.S. Pat. No. 9,645,075 titled “Multispectral imager with hybrid double layer filter array,” U.S. Pat. No. 9,395,473 titled “Nano-optic filter array based sensor,” U.S. Pat. No. 8,542,359

titled “Digital filter spectrum sensor,” U.S. Pat. No. 8,462,420 titled “Tunable plasmonic filter,” U.S. Pat. No. 8,330,945 titled “Multi-purpose plasmonic ambient light sensor and visual range proximity sensor,” U.S. Pat. No. 8,284,401 titled “Digital filter spectrum sensor,” and U.S. Pat. No. 8,274,739 titled “Plasmonic fabry-perot filter.” The entire contents of each of the above U.S. Patents are incorporated herein by reference. In one embodiment, each plasmonic filter has different transmission responses. In one embodiment, each plasmonic filter can have different ranges for full width half maximum of the transmission coefficient as a function of wavelength. In one embodiment, a predominant subset of the optical transmission responses of the plasmonic filters (i.e., a subset that includes at least one half of all optical transmission responses of the plasmonic filters) may have a respective transmission peak at a respective wavelength with a respective full width at half maximum in a range from 40 nm to 60 nm.

In one embodiment, the multiple peak wavelengths comprise at least two peak wavelengths within a wavelength range between 400 nm and 800 nm and at least one peak wavelength within an infrared wavelength range. In one embodiment, the total number of the plurality of filter-sensor assemblies 30 may be at least 9. For example, the total number of the plurality of filter-sensor assemblies 30 may be in a range from 9 (for example, by employing three peak wavelengths and three optical filters per peak wavelength are employed) to 4,096 (for example, by employing 32 peak wavelengths and 32 optical filters per peak wavelength are employed).

In one embodiment, the on-chip integrated biological sensing device 100 comprises an on-chip integrated photoplethysmography (MW-PPG) device. In one embodiment, the at least one biological measurement parameter comprises blood pressure. In one embodiment, the at least one biological measurement parameter comprises an oxygen saturation level in oxygen-carrying cells in the blood. In one embodiment, the light source assembly 20 is configured to emit the light continuously and to operate the plurality of filter-sensor assemblies 30 until a user input for termination of measurement is received or until a pre-programmed timer expires.

In one embodiment, the electronic package is configured to interface with, and to provide human-machine interface through, a host computing device through a universal serial bus (USB) connector 60 or a wireless communication module located within the electronic package and configured to communicate with a dongle having a USB connector and configured to be attached to the host computing device. In one embodiment, the automated program is configured to generate the measurement value for the at least one biological measurement parameter through calculations performed within the embedded processor (i.e., the processor located within the electronic package), or through calculations performed in an external processor in the host computing device (such as the CPU of a personal computer or a cellular phone), or through calculations performed in a server (that is connected to the host computing device via the internet) employing electronic transmission of the measured spectral distributions and electronic receipt of the at least one parameter as calculated by the server.

In one embodiment, the electronic package comprises: a communication module that is configured to communicate with the host computing device; and the automated program is configured to display instructions for operation of the on-chip integrated biological sensing device 100 to a user on the display unit of the host computing device, or to display instructions for downloading a program for operation of the on-chip integrated biological sensing device 100 on the display unit of the host computing device.

In one embodiment, the automated program is configured to run a maximum-ratio-combined (MRC) algorithm on the measured spectral distributions from the plurality of filter-sensor assemblies 30 and to generate the measurement value for the at least one biological measurement parameter employing the embedded processor or the external processor. In one embodiment, the electronic package is configured to be powered by the host computing device upon connection of to the host computing device, for example, through a USB connection (60, 62).

The on-chip integrated biological sensing device 100 of the present disclosure can be operated to measure a heartbeat rate, blood pressure, and/or an oxygen saturation level in oxygen-carrying cells of a person that is/are calculated from the maximal-ratio combined PPG signals as the at least one biological measurement parameter.

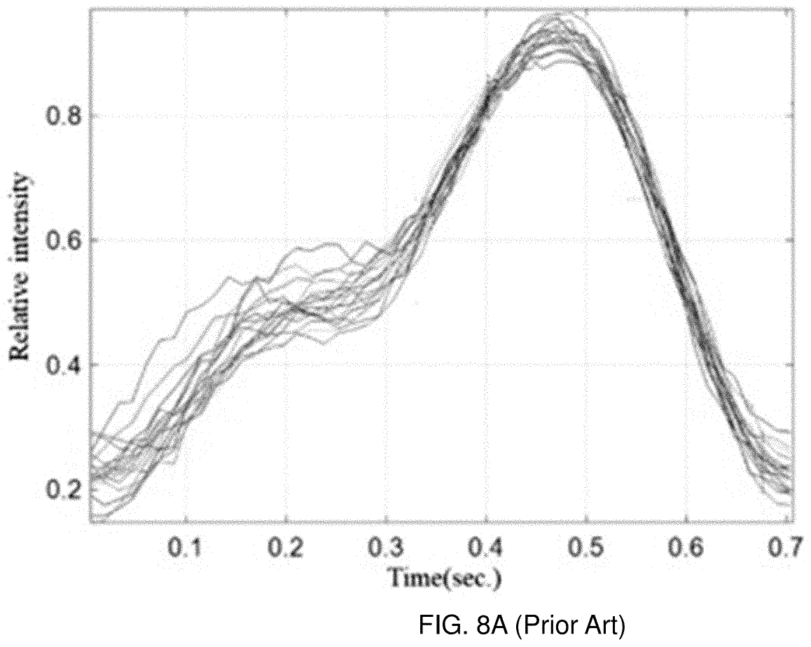

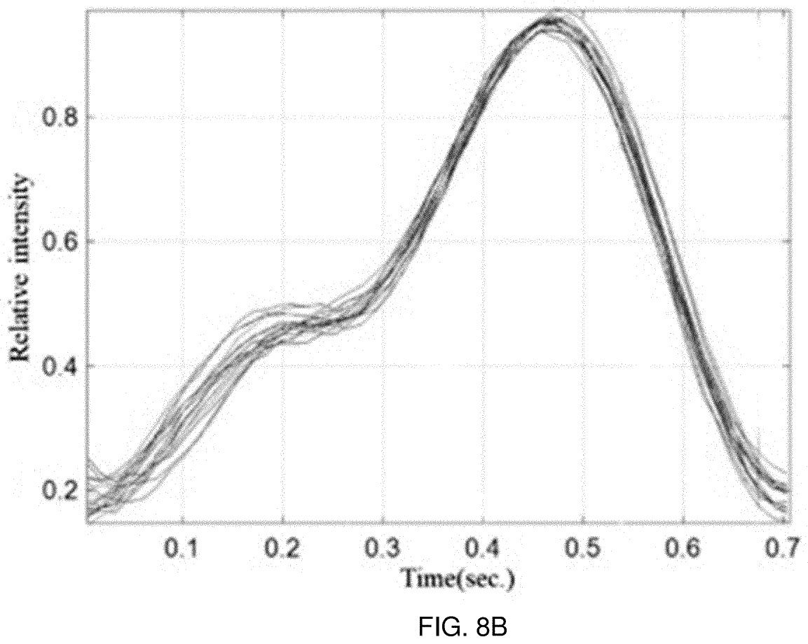

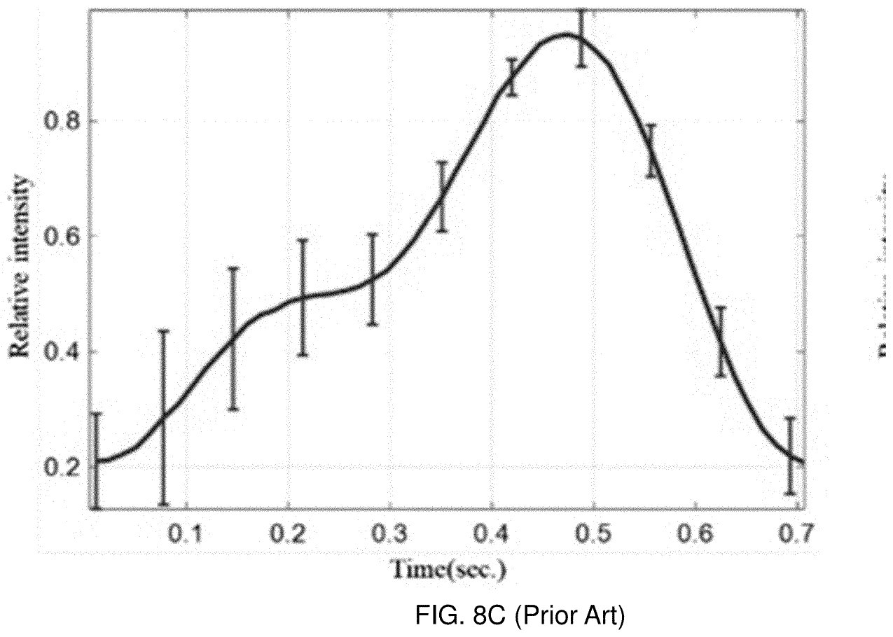

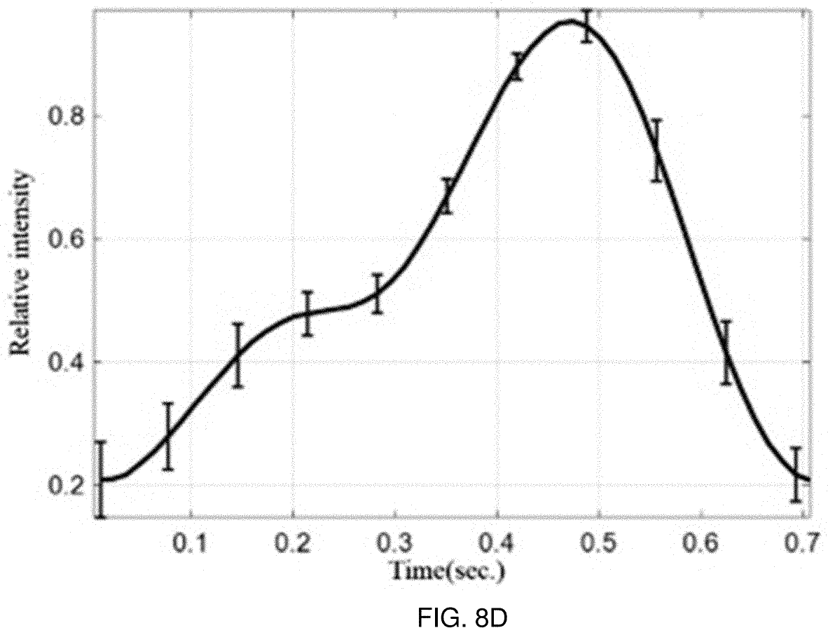

FIG. 8 shows the comparison of stability of the PPG signals from a subject using the integrated MW-PPG sensing device developed against the SW-PPG sensing device. The curves in FIG. 8A show the overlapped PPG waveforms collected using the reference SW-PPG sensing device (i.e., employing a prior art device illustrated in FIG. 1B). The curves in FIG. 8B show the overlapped PPG waveforms collected using the on-chip integrated MW-PPG sensing device of the present disclosure. For better clarity, to understand the variation of the SW-PPG waveforms at different time segments, the averaged curve and error bars (corresponding to the standard deviation at each measurement time) of the overlapped PPG waveforms in FIG. 8A are plotted in FIG. 8C. The averaged curve and error bars (corresponding to the standard deviation at each measurement time) of the overlapped MW-PPG waveforms in FIG. 8B are plotted in FIG. 8D. The average variation of SW-PPG signals was 0.142 as shown in FIG. 8C, whereas the average variation of the MW-PPG signals was only 0.077 as shown in FIG. 8D. Compared to the reference SW-PPG sensor, the integrated MW-PPG sensing device developed could effectively reduce the average variation by about 50%.

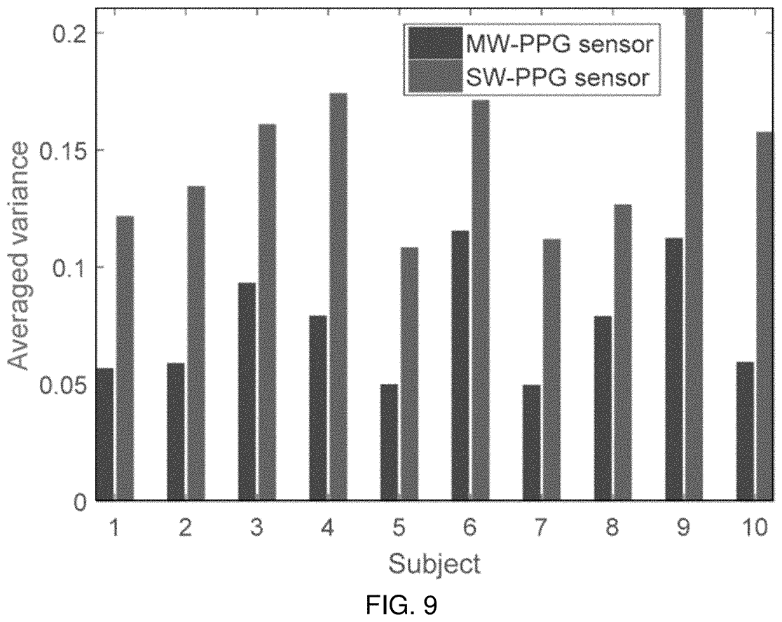

FIG. 9 shows the comparison of stability of the PPG signals among the 10 subjects, where the bars represent the averaged variation of PPG signals derived from the SW-PPG sensing device and that from the integrated MW-PPG sensing device, respectively. It shows that, in general, compared to the SW-PPG sensing device, around 50% variation reduction could be obtained in using the developed integrated MW-PPG sensing device.

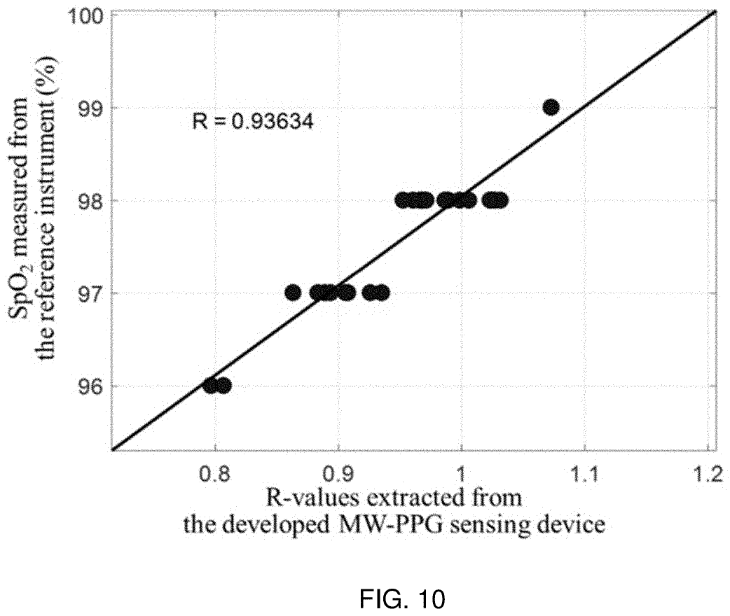

FIG. 10 shows the correlation between SpO₂ and R-values extracted from the integrated MW-PPG sensing device developed, where the x-axis represents the R-values extracted from the integrated MW-PPG sensing device and the y-axis represents the SpO₂ measured by the reference instrument. From the preliminary experimental results, it can be seen that the R-value against the SpO₂ value can deliver a high correlation coefficient up to 0.93, which matches the experimental result reported in. It shows the potential of SpO₂ measurement using the integrated MW-PPG sensing device developed.

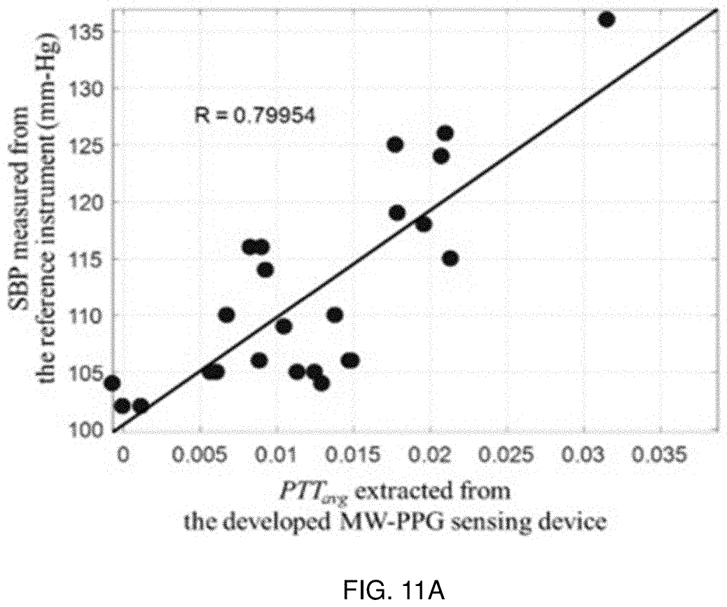

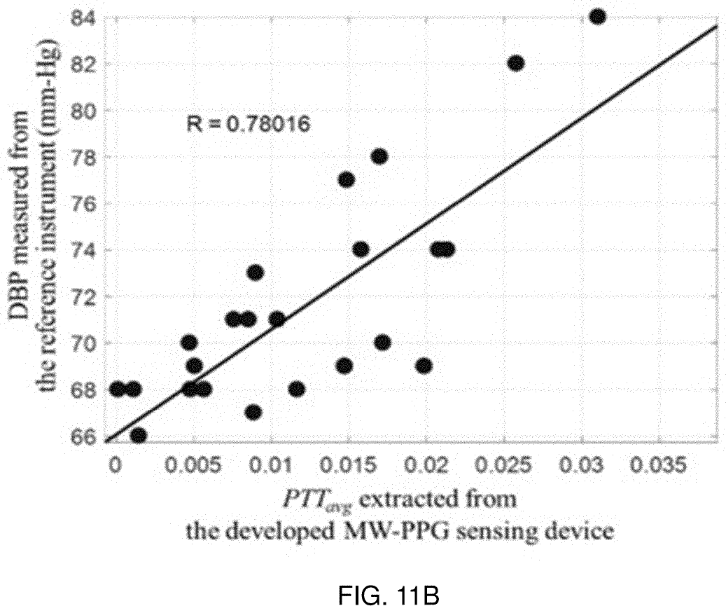

FIGS. 11A and 11B show the correlation between SBP and DBP, against PTTavg which is extracted from the integrated MW-PPG sensing device developed. In FIG. 11A, the x-axis represents the PTT_{avg} and the y-axis represents SBP. In FIG. 11B, the x-axis represents the PTT_{avg} and the y-axis represents DBP. The approach disclosed herein can enable an innovative chip-scale integrated MW-PPG sensing device for synchronously sensing MW-PPG signals. To quickly assess the potential capabilities of the device developed, rather than conducting a comprehensive medical case study which involves larger-scale budgets and time, only a simple correlation analysis was conducted as for pre-screening. Correlation coefficients R=0.79 between PTTavg and SBP, and correlation coefficients R=0.78 between PTT_{avg} and DBP were observed. The PTT_{avg} extracted from the integrated MW-PPG sensing device developed show a sufficient high correlation on blood pressure. The PTT_{avg} extracted from the integrated MW-PPG sensing device was useable to estimate SBP and DBP via a simple linear regression model.

As mentioned above, the current available PPG sensing devices on the market are not in the structure of synchronous MW-PPG sensing, and the main functionality is for heart rate detection. Our innovative, chip-scale and fully-on-chip integrated may MW-PPG sensing devices not only have the potential to provide a more stable and robust PPG signals, from the benefits of the MRC signal combining algorithm, for accurate heart rate detection, but also can simultaneously provide both R-values, for SpO₂ detection, and PTT_{avg}, potentially for blood pressure detection.

Generally, analysis of color of a biological sample employing a conventional color sensor is insufficient for generation of biological data for PPG because such data requires comparison of multiple spectral distributions of light passing through different filters for each peak wavelength in a light source assembly. The chip-scale integrated MW-PPG sensing device of the present disclosure analyzes the spectral distribution of reflected light from a biological sample employing a plurality of plasmonic filters such as at least three or more plasmonic filters per peak wavelength within a multi-peak light spectrum emitted from the light source assembly. In one embodiment, the on-chip integrated MW-PPG sensing device of the present disclosure can employ at least three different peak wavelengths for illumination, and for each peak wavelength, at least three different plasmonic filters cam be employed to further discriminate the spectral distribution of light from the biological sample to a level that enables extraction of biological data from the reflected light from the biological sample. A test sample for the chip-scale integrated MW-PPG sensing device employed three different peak wavelengths for illumination, and employed 5 different optical filters for each peak wavelengths, thereby measuring the reflected light with 15 different optical filters. Combined with an algorithm for correlating the spectral distribution of reflected light with PPG parameters, the multiple optical filters sufficiently granulated data collection on the spectral distribution of light and enables PPG measurements. Thus, blood pressure and SpO₂ measurements are possible employing the on-chip integrated MW-PPG sensor device of the present disclosure.

If compared to the sequential sampling architecture currently available on the market, the integrated MW-PPG sensing device developed is capable of synchronously sampling PPG signals of a large number wavelengths from a full-wavelength LED or few single-wavelength LEDs. If compared to conventional spectrometers, such as using Ocean Optics STS Microspectrometer™ (model: STS-VIS), used by the early researchers for constructing primitive MW-PPG measurement platforms with synchronous sampling architecture, the integrated MW-PPG sensing device developed can provide a competitive advantage in size and cost for daily applications.

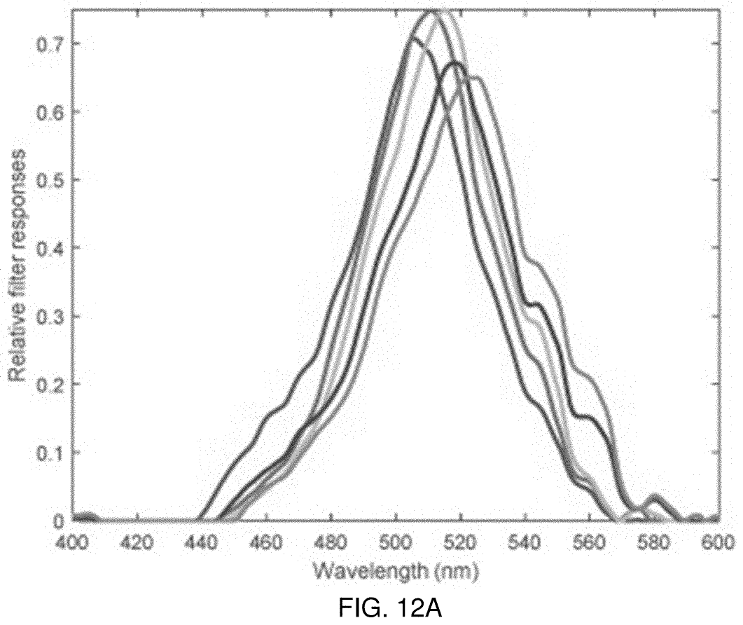

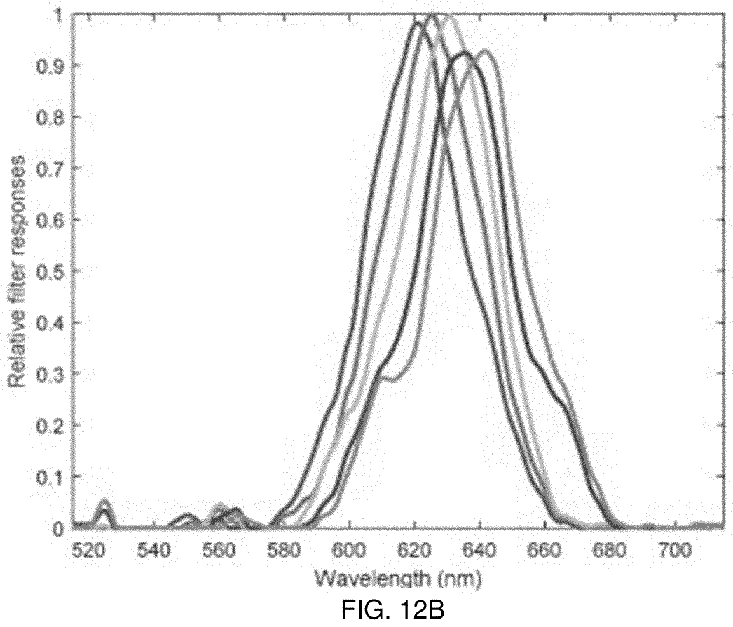

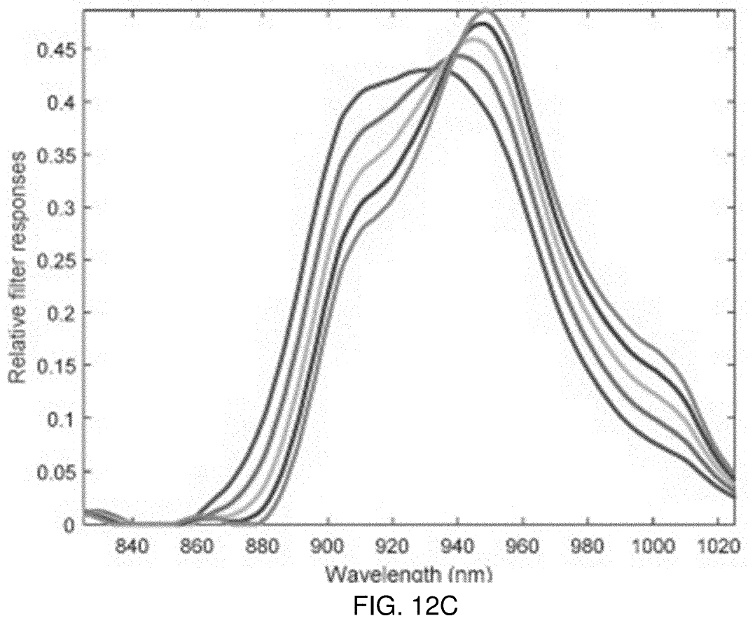

The filter responses of the integrated MW-PPG sensing device developed are shown in FIGS. 12A, 12B, and 12C. Peaks in the filter response (i.e., the transmission response) are located at 505 nm, 510 nm, 515 nm, 520 nm, 525 nm, 620 nm, 625 nm, 630 nm, 635 nm, 640 nm, 930 nm, 935 nm, 940 nm, 945 nm, and 950 nm, with full width at half maximum (FWHM) around 40˜60 nm. While achieving the channel selection purpose, cross-talk among adjacent channels is unavoidable since the pass bands of the filters are broad and overlapped. However, the side-lobes of the pass bands of the filters are well suppressed, so that a full-wavelength light source assembly or few single-wavelength LEDs can be used. 15 PPG signals corresponding to these regions of different wavelengths can then be acquired. The wavelengths of the light source assembly picked need to cover the sensitivity region of the implemented filters. In a demonstration of the integrated multi-wavelength biological sensing device, to focus on demonstrating applications of SpO₂ measurement and blood pressure measurement, LEDs of blue green, and the IR region were implemented as for a practical and cost-efficient implementation. Generally, multiple plasmonic filters (such as 3-16 plasmonic filters) can be employed for each peak wavelength in the multi-peak light spectrum that is irradiated onto a biological sample.

In the exemplary on-chip integrated MW-PPG sensing device described above, three spectral regions centered at 515 nm, 630 nm and 940 nm were used to synchronously obtain 15 PPG signals corresponding to these regions of different wavelengths employing cost-effective plasmonic filters. By utilizing the maximal-ratio combined (MRC) algorithm, the calculated biological parameters showed a reduction of about 50% in variations compared to the variations that are obtained employing the single-wavelength reference sensor. Besides, both the R-values for the SpO₂ measurement by using the red and infrared regions, and the pulse transit time (PTT) for the blood pressure measurement by using the green and infrared regions were investigated. The correlation coefficient between the R-values and the SpO₂ could be as high as R=0.93. The correlation coefficients between the PTT against systolic blood pressure (SBP) and diastolic blood pressure (DBP) could reach R=0.79 and R=0.78, respectively. The integrated MW-PPG sensing device developed has full potential not only in conventional PPG measurement and SpO₂ measurement, but also in emerging blood pressure measurement for wearable devices, all in a synchronous and simultaneous manner.

Although the foregoing refers to particular preferred embodiments, it will be understood that the invention is not so limited. It will occur to those of ordinary skill in the art that various modifications may be made to the disclosed embodiments and that such modifications are intended to be within the scope of the invention. All of the publications, patent applications and patents cited herein are incorporated herein by reference in their entirety.

What is claimed is:

1. An on-chip integrated multi-wavelength biological sensing device comprising an electronic package, wherein the electronic package comprises:
a light source assembly configured to emit light having multiple peak wavelengths simultaneously toward a biological sample;
a plurality of filter-sensor assemblies configured to simultaneously measure spectral distribution of incident light that impinges from the biological sample, wherein each of the filter-sensor assemblies comprises a respective optical filter providing a respective optical transmission response and a respective optical sensor;
an embedded processor and a memory unit embedded within at least one semiconductor chip, wherein the memory unit stores an automated program configured to compile the measured spectral distribution of the incident light and to generate a measurement value for at least one biological measurement parameter pertaining to the biological sample; and
an enclosure containing the light source assembly, the plurality of filter-sensor assemblies, and the semiconductor chip.

2. The on-chip integrated multi-wavelength biological sensing device of claim 1, further comprising a circuit board to which the light source assembly, the plurality of filter-sensor assemblies, the embedded processor, and the memory unit are attached.

3. The integrated multi-wavelength biological sensing device of claim 1, wherein the plurality of filter-sensor assemblies is configured to synchronously measure the spectral distributions for each of the filter-sensor assemblies.

4. The integrated multi-wavelength biological sensing device of claim 1, wherein the light source assembly comprises a plurality of light emitting diodes configured to emit light of different peak wavelengths.

5. The integrated multi-wavelength biological sensing device of claim 4, wherein a total number of the plurality of filter-sensor assemblies is greater than a total number of the different peak wavelengths of the plurality of light emitting diodes at least by a factor 3.

6. The integrated multi-wavelength biological sensing device of claim 4, wherein:
the electronic package comprises at least one emission window pane and a reception window pane;
the plurality of light emitting diodes is configured to emit light simultaneously through a respective one of the at least one emission window pane; and
the plurality of filter-sensor assemblies is configured to receive light through the reception window pane.

7. The integrated multi-wavelength biological sensing device of claim 6, wherein:
the electronic package comprises at least as many emission window panes as a total number of peak wavelengths among the multiple peak wavelengths;
each light emitting diode among the plurality of light emitting diodes is configured to emit light through different emission window panes; and
the emission window panes are arranged around the reception window pane on a front side of the electronic package.

8. The on-chip integrated multi-wavelength biological sensing device of claim 1, wherein the optical filters within the plurality of filter-sensor assemblies comprise plasmonic filters including a respective metallic film containing nanoscale structures

9. The on-chip integrated multi-wavelength biological sensing device of claim 8, wherein each plasmonic filter has different transmission responses.

10. The integrated multi-wavelength biological sensing device of claim 1, wherein the multiple peak wavelengths comprise at least two peak wavelengths within a wavelength range between 400 nm and 00 nm and at least one peak wavelength within an infrared wavelength range.

11. The integrated multi-wavelength biological sensing device of claim 1, wherein the total number of the plurality of filter-sensor assemblies not less than 9.

12. The integrated multi-wavelength biological sensing device of claim 1, wherein the integrated multi-wavelength biological sensing device comprises an on-chip integrated photoplethysmography (MW-PPG) device.

13. The integrated multi-wavelength biological sensing device of claim 1, wherein the integrated multi-wavelength biological sensing device automated program employs maximum-ratio-combined (MRC) algorithm to extract maximal-ratio combined photoplethysmography (PPG) signals from raw PPG signals from the optical filters.

14. The integrated multi-wavelength biological sensing device of claim 13, wherein the at least one biological measurement parameter comprises a heartbeat rate that is calculated from the maximal-ratio combined PPG signals.

15. The integrated multi-wavelength biological sensing device of claim 13, wherein the at least one biological measurement parameter comprises blood pressure that is calculated from the maximal-ratio combined PPG signals.

16. The integrated multi-wavelength biological sensing device of claim 13, wherein the at least one biological measurement parameter comprises an oxygen saturation level in oxygen-carrying cells in the blood that is calculated from the maximal-ratio combined PPG signals.

17. The integrated multi-wavelength biological sensing device of claim 1, wherein the electronic package is configured to interface with, and to provide human-machine interface through, a host computing device, through a universal serial bus (USB) connector or a wireless communication module located within the electronic package

18. The integrated multi-wavelength biological sensing device of claim 17, wherein the automated program is configured to generate the measurement value for the at least one biological measurement parameter through calculations performed within the embedded processor, or through calculations performed in an external processor in the host computing device, or through calculations performed in a server employing electronic transmission of the measured spectral distributions and electronic receipt of the at least one parameter as calculated by the server.

19. The integrated multi-wavelength biological sensing device of claim 1, wherein the electronic package comprises:
a communication module that is configured to communicate with the host computing device; and
the automated program is configured to display instructions for operation of the integrated multi-wavelength biological sensing device to a user on the display unit of the host computing device, or to display instructions for downloading a program for operation of the integrated multi-wavelength biological sensing device on the display unit of the host computing device.

20. A method of operating the integrated multi-wavelength biological sensing device of claim 1, comprising:
providing the integrated multi-wavelength biological sensing device of claim 1; and
measuring a heartbeat rate, blood pressure, or an oxygen saturation level in oxygen-carrying cells of a person as the at least one biological measurement parameter.
